(19) United States
(12) Patent Application Publication
(10) Pub. No.: US 2006/0264794 A1
(43) Pub. Date: Nov. 23, 2006

(54) Improved Cuff for Securing Objects to Cuffed Object

(76) Inventors: Leila Sue Fuchs, Bellevue, WA (US); Rose Dorothy Fuchs, Grand Forks, ND (US)

Correspondence Address: RONALD GRANT, 15600 NE 8TH ST, SUITE B1 #304, BELLEVUE, WA, 98008, US

(21) Appl. No.: 10/908,689

(22) Filed: May 23, 2005

(51) Int. Cl. A61F 5/00

(52) U.S. Cl. 602/26

(57) ABSTRACT

The present invention is an improved means of variable size and of variable length with an enclosure means to accommodate an object to be cuffed. The improved cuff has space for one or more opening means through which the object being cuffed can be accessed. The outer surface of the improved cuff is made from a material that can accommodate adhesive or other securing means, such as hook and loop fastening. Items that need to be secured to the object being cuffed can, instead, be secured using the surface of the improved cuff.

### CROSS-REFERENCE TO RELATED APPLICATIONS

None

### FEDERALLY SPONSORED RESEARCH

None

### SEQUENCE LISTING OR PROGRAM

None

### BACKGROUND OF THE INVENTION

1. Field of Invention

This specification describes an improved cuff that provides access to areas underneath the cuff. The cuff provides a surface to attach, adhere or apply an adhesive material to the surface of the cuff rather than the object the cuff encloses.

2. Objects and Advantages

The concept of such an improved cuff is best exemplified in the field of medical procedures.

In the art, the focus of securing objects to patients centers on the medical device to be secured. See, for example, U.S. Pat. No. 3,722,508. This cuff does its job well—secure the IV to the patient. However, the design of such an IV centered cuff tends to lack concern for the comfort or anxiety of the patient.

Additionally, the common routine for securing an IV to the skin is to hold the IV onto a patient using adhesive tape. This is despite the fact that many patients requiring medical procedures cannot have latex or adhesive materials placed on their skin. This may be due to allergies or other skin reactions.

An accommodating and simple cuff designed to be applied in a simple manner would accommodate both the needs of the medical practitioner (secure a medical device using routine procedures, such as securing an IV with adhesive tape) and the needs of the patient (to feel comfortable and without incurring further discomfort through a reaction to the adhesive).

### SUMMARY

The present invention is a cuff means of variable size and of variable length with an enclosure means to accommodate an object to be cuffed. The improved cuff has space for one or more opening means through which the object being cuffed can be accessed. The improved cuff is made of a material that can accommodate adhesive or other securing means, such as hook and loop fastening. This allows items to access the object being cuffed and allows such items to be secured onto the improved cuff without touching the object being cuffed.

These and other features and advantages of the present version of the invention will become better understood with regard to the following description, appended claims and accompanying drawing(s).

### DRAWINGS

FIG. 1 is a top down view of the improved cuff.

### DETAILED DESCRIPTION

Definition List 1

| Term | Definition |
|---|---|
| cuff | Websters dictionary defines a as a bandlike material. |
| | A band is defined as a thin strip of flexible material |
| | used to encircle and bind one object. In this |
| | specification, the word cuff encapsulates the bandlike |
| | qualities referred to in the Webster's definition of |
| | a band. That is, a cuff is a thin strip of flexible |
| | material used to encircle and bind an object. |

Preferred Embodiment:

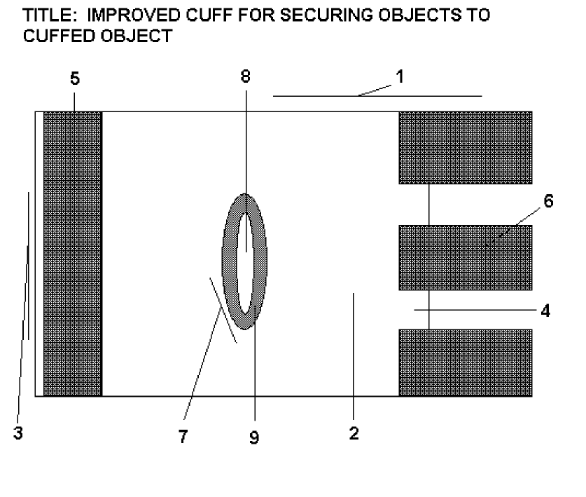

A preferred embodiment of this version of the invention is described in FIG. 1.

FIG. 1 is an overhead view of the top side of the improved cuff 1. The improved cuff 1 is made from a material 2 that can be elastic in nature. The size of the improved cuff 1 depends upon the size of the object to be cuffed. In the preferred embodiment of this version of the invention, illustrated in FIG. 1, the material is cut to accommodate a typical human arm. That is, the material 2 is cut in a rectangular pattern. The length is cut to accommodate the circumference of an arm and the width is cut to accommodate strips of adhesive tape above and below the opening means 7.

To secure the improved cuff 1 to a body, there are adjustable fastening means attached to each end (3, 4) of the material. In the preferred embodiment, attached to the material 2 at one end 3 is a strip of hook fabric 6 to accommodate hook and loop fastening material. At the opposite end 4 is attached one or more strip(s) of loop fabric 6, designed to be fastened on the opposing hook fabric 5. Whether the hook material or loop material is attached to a specific end is not relevant, and equivalent fastening means are claimed. In the preferred embodiment there are three loop fabric straps 6 that are fastened so as to be perpendicular to the end 4 of the cuff material 2.

The preferred embodiment disclosed in this version of the invention has a single opening means 7 with a pre-sewn hem 9 to secure the fabric around the opening 8.

The improved cuff 1 can be comprised of material 3, securing means 4, 5, and opening means 7 so that it is to be disposed after a single use. The improved cuff 1 can be can be comprised of material 3, securing means 4, 5, and opening means 7 so that it can be reused after each use.

The preferred embodiment of this version of the invention is for securing IVs in a comfortable manner.

The improved cuff 1 is secured around the arm of a patient with the opening means 7 centered so that the area of the opening 3 is placed on the area of the patient's arm that needs to be accessed.

Securing strips 6 are placed so as to secure the improved cuff around the patient's arm by securing said securing strip(s) 6 to the opposing strip 5.

The material 2 in the preferred embodiment disclosed in this version of the invention is elastic enough to stretch and comfortably accommodate a patient's arm. When the securing ends 5, 6 are closed and tightened to a point where the improved cuff 1 is secured to the patient, the opening means 7 of the improved cuff should not move away from the area where the IV is to be inserted.

The IV is inserted through the opening 8 of the opening means 7.

Adhesive tape, used to secure the IV, is then secured to the cuffed object by applying the adhesive tape to the improved cuff 1. This allows the IV to be secured to the patient so that the adhesive tape is not touching the patient's skin.

It is understood that this version of the improved cuff is not limited to the particular construction and arrangement of parts herein illustrated and described, but embraces all such modified forms thereof as come within the scope of the following claims.

What is claimed is:

1. An improved cuff comprising:
a) a material with an outer surface sufficient to accommodate securing one or more adhesive means onto said material;
b) said material being a width sufficient to surround the object to be bound, i.e., cuffed;
c) the ends of said material having an enclosure means sufficient to fasten and secure the improved cuff so that the improved cuff is held securely around the object to be cuffed; and
d) said improved cuff being a length sufficient to accommodate at least one opening means through said material.

2. The improved cuff of claim 1 wherein the said opening means is at least one hemmed opening in the material of the improved cuff.

3. The improved cuff of claim 1 wherein the improved cuff is made from a material designed to be disposed after a single use.

4. The improved cuff of claim 1 wherein the improved cuff is made from a material that can be cleaned and re-used after each use.

5. A method for using an improved cuff comprising the steps of:
a) placing a cuff around an object to be cuffed;
b) securing opposing ends of the cuff so that the cuff is held securely around the object to be cuffed;
c) an opening means in the cuff is opened;
d) an object is placed through the opening means; and
e) an attaching means is used to secure said object to the cuffed object by attaching said attaching means to said cuff, without said attaching means making contact with the cuffed object.

6. The method for using an improved cuff, as claimed in claim 3, wherein the object to be secured to the cuffed object is an IV (a device used to administer a substance into a vein) and the attaching means used to secure said IV is adhesive tape attached to the outer surface of the improved cuff.

7. The method for using an improved cuff, as claimed in claim 3, wherein the opening means in the cuff is opened by using a cutting means.
